(19) United States
(12) Patent Application Publication
(10) Pub. No.: US 2007/0020302 A1
(43) Pub. Date: Jan. 25, 2007

(54) Exfoliating Cream

(75) Inventor: Eckart Mathias, Goleta, CA (US)

Correspondence Address: CROWELL & MORING LLP;INTELLECTUAL PROPERTY GROUP, P.O. BOX 14300, WASHINGTON, DC, 20044-4300, US

(73) Assignee: Den-Mat Corporation, Santa Maria, CA (US)

(21) Appl. No.: 11/183,959

(22) Filed: Jul. 19, 2005

(51) Int. Cl. A61K 8/02

(52) U.S. Cl. 424/401

(57) ABSTRACT

An exfoliating cream and method of forming. The cream includes particles of a stone. The stone may be a precious or semi-precious stone of a characteristic color. The cream appears to be the color of the characteristic color. The cream may also include coloring agents to impart the characteristic color. The cream may also include particles of barium glass as another exfoliant. The cream may also include whole stone cells as another exfoliant.

### FIELD OF THE INVENTION

The present invention relates to exfoliating systems.

### BACKGROUND OF THE INVENTION

Skin care has long been important to many people. This is especially true of facial skin care. Creams, lotions, and cleansers are continually being developed in an attempt to supply a product which provides fresher healthier softer skin. Cleansers which clean the skin, moisturizers which soften, and exfoliates which attempt to remove dead skin cells are all used to try to reach this goal.

Cleansers are used to remove skin oils, dirt, and other foreign material from the skin. Soap, one of the earliest cleansers is quite successful in removing foreign material and oils from the skin. However, soap also tends to dry the skin. There have been many attempts to rectify this problem by adding moisturizers to soap in an attempt to keep skin soft and supple. Many additives to soap products clog skin pores resulting in various problems. After a cleanser is used, a moisturizer is often used in an attempt to soften and condition the skin. While moisturizers can condition dead skin cells, they cannot restore them or improve growth of new skin below the dead skin layers.

Commonly, an exfoliant is used in an attempt to remove dead skin cells exposing the living layer. This provides the skin a smoother softer more youthful appearance, as well as allowing the living layers to grow and stay healthy. Exfoliating creams conventionally use an abrasive compound in a scrubbing motion to remove dead skin cells from the face. Common exfoliants include powders from ground apricot seeds, crushed walnut shells, coconut shells, almond seeds and shells, and sawdust, various solid polymer powders, and various inorganic particles such as sand, salt, alumina, silica, alumino-silicates, lava stone, various phosphates, borates, sulfates, and carbonates.

U.S. Pat. No. 6,432,430, issued to Fitzjarrell, teaches an exfoliating cream containing crushed walnut shells as an exfoliant. U.S. Pat. No. 6,290,976, issued to Messenger, discloses an exfoliating cream comprising corundum or aluminum oxide particles as exfoliants.

It has also been proposed that crushed precious stones could be used as exfoliants. For example, U.S. Pat. No. 6,036,965, issued to Gubernick et al., discloses a cleanser which includes ground tourmaline as an exfoliant. Similarly, U.S. Patent Application No. 2004/0091446, credited to Massaro et al., discloses a liquid cleaning composition comprising exfoliants. A long list of possible exfoliants disclosed includes certain precious stones such as amethyst powder, diamond powder, emerald, and topaz.

U.S. Patent Application 2003/016550, credited to Rhoades, discloses a composition comprising a base and abrasive or exfoliating particles. The composition is designed for use with an application device that via vibration or rotation physically manipulates the abrasive particles against the skin. Suitable particles include precious stones such as diamond, garnet, sapphire, ruby, emerald, and topaz.

However, none of these references teach an exfoliating cream comprising particles of a stone of a characteristic color wherein the cream appears to be the color of the whole stone. Further, none of these references teach an exfoliating cream comprising fluorine-containing particles. Further, none of these references teach an exfoliating cream comprising whole stone cells or sclereids.

### SUMMARY OF THE INVENTION

In a first embodiment of the invention, an exfoliating cream includes a base cream and exfoliating agents. The exfoliants include particles of stone. The virgin stone has a characteristic color. The cream exhibits this characteristic color.

In a second embodiment of the invention, an exfoliating cream includes a base cream and exfoliating agents. The exfoliating agents include particles of a fluorine-containing barium glass and/or unground stone cells.

In a third embodiment of the invention, a method for forming an exfoliating cream includes providing a base cream, providing particles of stone, and providing at least one coloring agent. The color of the virgin stone is determined. At least one coloring agent is chosen based on this color determination in order to match the color.

### DETAILED DESCRIPTION OF THE INVENTION

For simplicity and illustrative purposes, the principles of the present invention are described by referring to various exemplary embodiments thereof. Although the preferred embodiments of the invention are particularly disclosed herein, one of ordinary skill in the art will readily recognize that the same principles are equally applicable to, and can be implicated in other compositions and methods, and that any such variation would be within such modifications that do not part from the scope of the present invention. Before explaining the disclosed embodiments of the present invention in detail, it is to be understood that the invention is not limited in its application to the details of any particular embodiment shown, since of course the invention is capable of other embodiments. The terminology used herein is for the purpose of description and not of limitation. Further, although certain methods are described with reference to certain steps that are presented herein in certain order, in many instances, these steps may be performed in any order as may be appreciated by one skilled in the art, and the methods are not limited to the particular arrangement of steps disclosed herein.

One embodiment of the present invention is drawn towards an exfoliating cream. The exfoliating cream contains a base cream and particles of a ground stone. The virgin or whole stone has a characteristic color. The cream exhibits this characteristic color. The color may be provided by the natural color of the ground stone. Alternatively, the color may be provided with coloring agents added to the cream to substantially match the characteristic color of the stone.

The base cream may be any common skin cream such as a cleansing or moisturizing cream. The base cream may contain additives such as aloe and vitamins. The base cream may have cleansing or moisturizing features. When desired, the base cream may qualify as both a cleanser and a moisturizer cream. Such creams may contain common ingredients used by those skilled in the art, and may include compounds such as cetyl alcohol, sorbitan stearate, propylene glycol, stearic acid, triglyceride, mineral oil, allantoin, aloe, polysorbate, sorbitol, water, parabens, vitamins, fragrances, and colorants.

Preferably, the stone is a precious or semi-precious stone. More preferably, the stone is selected from the group consisting of topaz, emerald, amethyst, sapphire, agate, ruby, citrine, garnet, aquamarine, carnelian, fire opal, Peruvian opal, and turquoise. The coloring agent can be any suitable compound to impart the color of the stone to the cream. For example, ruby has a characteristic deep red color. Red coloring agents could be added to the cream to impart a red color to the cream. The stones can be ground into particles by any conventional method for use in the cream. A list of stones, their characteristic color, and examples of color matching additives can be found in Table 1.

TABLE 1

| | | Stone | Examples of |
|---|---|---|---|
| | Typical | Matching Color | Matching Color |
| Stone | Color | Additive(s) | Additives |
| Sapphire | Blue | Blue | FD&C Blue#1, |
| | | | FD&C Blue#2. |
| Topaz | Amber | Yellow, | FD&C Yellow#5, |
| | | red, blue | FD&C Yellow#6, |
| | | | D&C Yellow#10, |
| | | | FD&C Red#3, FD&C |
| | | | Red#40, FD&C |
| | | | Blue#1, FD&C |
| | | | Blue#2. |
| Topaz | Blue | Blue | FD&C Blue#1, |
| | | | FD&C Blue#2. |
| Ruby | Red | Red | FD&C Red#3, FD&C |
| | | | Red#40, FD&C |
| | | | Blue#1. |
| Emerald | Green | Green, blue, | FD&C Green#3, |
| | | yellow | FD&C Blue#1, |
| | | | FD&C Blue#2, |
| | | | FD&C Yellow#5, |
| | | | FD&C Yellow#6, |
| | | | D&C Yellow#10. |
| Citrine | Yellow | Yellow, | FD&C Yellow#5, |
| | | red, green, | FD&C Yellow#6, |
| | | blue | D&C Yellow#10, |
| | | | FD&C Red#3, FD&C |
| | | | Red#40, FD&C |
| | | | Green#3, FD&C |
| | | | Blue#1, FD&C |
| | | | Blue#2. |
| Amethyst | Purple | Blue, red | FD&C Blue#1, |
| | | | FD&C Blue#2, |
| | | | FD&C Red#3, FD&C |
| | | | Red#40. |
| Garnet | Burgundy | Red, blue, | FD&C Red#3, FD&C |
| | red | yellow | Red#40, FD&C |
| | | | Blue#1, FD&C |
| | | | Blue#2, FD&C |
| | | | Yellow#5, FD&C |
| | | | Yellow#6, D&C |
| | | | Yellow#10. |
| Aquamarine | Light blue | Blue | FD&C Blue#1, |
| | | | FD&C Blue#2. |
| Carnelian | Reddish-orange | Red, yellow | FD&C Red#3, FD&C |
| | | | Red#40, FD&C |
| | | | Yellow#5, FD&C |
| | | | Yellow#6, D&C |
| | | | Yellow#10. |
| Fire opal | Red/pink | Red, blue | FD&C Red#3, FD&C |
| | | | Red#40, FD&C |
| | | | Blue#1, FD&C |
| | | | Blue#2. |
| Peruvian opal | Blue | Blue | FD&C Blue#1, |
| | | | FD&C Blue#2. |
| Turquoise/Teal | Blue-green/ | Blue, green, | FD&C Blue#1, |
| | Green-blue | yellow | FD&C Blue#2, |
| | | | FD&C Green#3, |
| | | | FD&C Yellow#5, |
| | | | FD&C Yellow#6, |
| | | | D&C Yellow#10. |

The exfoliating cream may also contain ground barium glass or other particles. The ground barium glass could be added to the cream in addition to the ground stone as another exfoliant. The ground barium glass may be the primary exfoliant The barium glass may also contain fluorine. An exemplary glass comprises barium, fluorine, aluminum, and silicon.

The fluorine-containing barium glass used in the exfoliating cream of the present invention can be prepared by the methods described in U.S. Pat. No. 5,360,770, issued to Chadwick, which is herein incorporated by reference in its entirety. The barium glass can be ground and subsequently sieved to a variety of particle sizes, covering size fractions of from about 5 to about 700 microns. Preferably, particle diameters of the glass in the cream range from about 40 to about 500 microns. More preferably, the particle diameters range from about 74 to about 420 microns. Preferably, the barium glass comprises barium, fluorine, aluminum, and silicon. Heretofore, such a glass will be referred to by the acronym BaFAlSi.

In addition to the barium glass, or alternatively, the exfoliating cream may also contain natural and stained stone cells or sclereids. The stone cells may be isolated from certain fruits such as pears, quince, and chico fruit. The stone cells may be isolated by any known method. Preferably, the stone cells may be used in a whole ungrounded state in the cream.

The stone cells may be colored with known stains, commonly used in photo microscopy, for chemically linking the colorant to the lignin of the cells. After such staining, the cells are washed to remove excess stain and to preclude color bleeding when the stone cells are formulated into a base cream. The size of the stone cells range from a minimum of about 40 microns to a maximum of about 750 microns, with most stone cells in the 350 micron range. The stone cells are substantially isodiametric, with some of the larger clusters of stone cells having aspect ratios of about 2 to 3. The stone cells may be used to impart a milder abrasiveness to the cream compared to the barium glass particles. Fragrances may also be added to the stone-cell-containing exfoliating cream to impart a distinct smell to the cream, such as that of a pear.

Another embodiment of the present invention is drawn towards a method of forming an exfoliating cream. The method includes providing a base cream, providing particles of stone, determining the color of the virgin stone, and providing at least one coloring agent to match the determined color. The color can be determined by visually inspecting the virgin stone or by consulting a reference on the stone.

Ground semi-precious or precious stones are good exfoliating agents. Users may also feel good about using an exfoliating cream that includes particles of their favorite gemstone. The likeness in color of the cream to the stone may reinforce this feeling.

There may be other benefits to using gemstones in skin cream. For example, some people believe the close proximity of such stones to one's body may have advantageous effects. These effects are taught by Traditional Chinese Medicine. Published U.S. Application No. 2003/0077238, credited to Roovers et al., which is herein incorporated by reference in its entirety, outlines effects of several gemstones. Whether these effects are real or only perceived, some users may desire to use an exfoliating cream that they believe may have greater effects than just exfoliation or moisturizing.

The use of the BaFAlSi glass particles in the cream may also have benefits. Fluorine is known to aid in healing carious tissue. Accordingly, fluorine may also have advantageous healing effects on damaged or worn skin, for example, the fluorine-releasing exfoliant may help prevent or heal the damaging effects of topical fungal and yeast infections on the skin. Users may also perceive fluorine to have positive effects and thus desire to use an exfoliating cream that contains fluorine.

Users may also desire to use a cream that contains botanical exfoliants, such as stone cells of natural origin. Users may feel more natural and connected to nature using such a cream compared to crushed seeds or shells. Users may also enjoy the milder abrasiveness imparted to the cream compared to creams with crushed seeds or shells.

The exfoliating cream of the present invention can be made by any well-understood method. For example, powder from the ground stone could be added to a common moisturizing or cleansing cream. Coloring agents could be added to the cream until the desired color is achieved. Ground barium glass could also be added to the cream if desired. Stone cells could be added to impart a natural mild exfoliating experience.

The base cream could be made by any common method and could contain any common compounds such as moisturizers, cleansers, and vitamins. The exfoliants and coloring agents could be added at any time in the process to the cream. The exfoliating cream could be prepared by a batch process or a continuous process.

Although creams containing precious or semi-precious stones have mainly been described, the present invention is not limited to such creams. The cream may contain any combination of crushed stone, ground barium glass, and whole stone cells. Accordingly, the present invention includes creams containing just barium glass as exfoliants, just whole stone cells as exfoliants, or crushed stone used in combination with either or both as exfoliants.

### EXAMPLES

### Example 1

### Formation of a Sapphire Blue Exfoliating Cream

Base Cream Formation:

To a 100 ml glass beaker, containing a magnetic stirring bar, was added 5.00 g cetyl alcohol, 5.00 g Polysorbate 60, 3.10 g stearic acid, 4.00 g caprylic/capric triglyceride, 3.00 g glycerol stearate, 3.00 g sorbitan stearate, and 1.50 g distearyldimethyl ammonium chloride. The mixture was heated to 85° C. and stirred until homogeneous. Into a separate 5 oz polypropylene (PP) beaker was added 12.00 g glycerin, and 62.00 g deionized water. This mixture was heated to about 72° C. and stirred vigorously using a disperser blade. Once both mixtures were up to temperature, the content of the glass beaker was added, continuously, slowly, and during vigorous mixing, to the content of the PP beaker. As the mixture became less fluid, the PP beaker was manually moved to engage the forming cream with the rotating high-speed blade until the cream was homogeneous. To this mixture was added 0.10 g methylparaben, 0.04 g propylparaben, and 1.26 g of Vitamin E, and the stirring of this mixture continued for about 10 minutes using a PP spatula. The cream was then allowed to cool to room temperature before use.

Staining of Stone Cells:

To 0.50 g of pear stone cells, in a small glass vial, was added 9.00 g of a 0.03% aqueous solution of Toluidine Blue O. The mixture was occasionally shaken for a total of about 70 minutes, after which it was allowed to settle, for repeated water washings by decantation. This was followed by two washings with isopropyl alcohol, also by decantation. The blue stones were then washed with acetone, to facilitate drying of the stones in a 40° C. oven.

Coloring of the Base Cream:

To a total of 86.00 g of base cream of Example 1, in a PP beaker, was added 0.0179 g of blue food coloring (FD&C Blue#1). The mixture was stirred thoroughly with a PP spatula, until the color is completely homogeneous.

Addition of Exfoliants to Colored Cream:

To 86.02 g of blue-colored cream, in a PP beaker, was added 1.72 g BaFAlSi (−100+200 mesh), 2.58 g BaFAlSi (−40+100 mesh), and 0.45 g of blue-stained stone cells. The mixture was stirred vigorously with a PP spatula, until homogeneous.

### Example 2

### Formation of a Ruby Red Exfoliating Cream

Base Cream Formation:

To a 250 ml glass beaker, containing a magnetic stirring bar, was added 20.00 g cetyl alcohol, 20.00 g Polysorbate 60, 12.40 g stearic acid, 24.00 g caprylic/capric triglyceride, 12.00 g glycerol stearate, 12.00 g sorbitan stearate, and 6.0 g distearyldimethyl ammonium chloride. The mixture was heated to 85° C. and stirred until homogeneous. Into a separate 1 L polypropylene (PP) beaker was added 48.00 g glycerin, and 240.00 g deionized water. This mixture was heated to about 72° C. and stirred vigorously using a disperser blade. Once both mixtures were up to temperature, the content of the glass beaker was added, continuously, slowly, and during vigorous mixing, to the content of the PP beaker. As the mixture became less fluid, the PP beaker was manually moved to engage the forming cream with the rotating high-speed blade until the cream was homogeneous. To this mixture was added 0.40 g methylparaben, 0.16 g propylparaben, and 5.04 g of Vitamin E, and the stirring of this mixture continued for about 10 minutes using a PP spatula. The cream was then allowed to cool to room temperature before use.

Staining of Stone Cells:

To 1.00 g of pear stone cells, in a small glass vial, was added 3.00 g of a 0.05% aqueous solution of Safranin O, and 1.00 g deionized water. The mixture was occasionally shaken for a total of about 70 minutes, after which it was allowed to settle, for repeated water washings by decantation. This was followed by two washings with isopropyl alcohol, also by decantation. The red stones were then washed with acetone, to facilitate drying of the stones in a 40° C. oven.

Coloring of the Base Cream:

To a total of 360.00 g of the base cream, in a PP beaker, was added 0.0421 g of red food coloring (FD&C Red#40). The mixture was stirred thoroughly with a PP spatula, until the color is completely homogeneous.

Addition of Exfoliants to Colored Cream:

To 80.00 g of the red-colored base cream, in a PP beaker, was added 1.69 g BaFAlSi (−100+200 mesh), 2.54 g BaFAlSi (−40+100 mesh), and 0.42 g of red stone cells. The mixture was stirred vigorously with a PP spatula, until homogeneous.

### Example 3

### Formation of an Emerald Green Exfoliating Cream

Base Cream Formation:

To a 100 ml glass beaker, containing a magnetic stirring bar, was added 2.00 g cetyl alcohol, 2.00 g Polysorbate 60, 3.10 g stearic acid, 1.00 g caprylic/capric triglyceride, 1.00 g glycerol stearate, 1.00 g sorbitan stearate, and 1.50 g distearyldimethyl ammonium chloride. The mixture was heated to 85° C. and stirred until homogeneous. Into a separate 5 oz polypropylene (PP) beaker was added 35.00 g glycerin, 6.00 g Laponite XLS, and 49.00 g deionized water. This mixture was heated to about 72° C. and stirred vigorously using a disperser blade. Once both mixtures were up to temperature, the content of the glass beaker was added, continuously, slowly, and during vigorous mixing, to the content of the PP beaker. To this still fluid mixture was added 0.10 g methylparaben, 0.04 g propylparaben, and 1.26 g of Vitamin E, and the stirring of this mixture continued for about another minute. The cream was then allowed to cool to room temperature before use.

Staining of Stone Cells:

To 0.403 g of quince stone cells, in a small glass vial, was added 1.210 g of a 0.03% aqueous solution of Toluidine Blue 0, and some extra deionized water to ease mixing. The mixture was occasionally shaken for a total of about 70 minutes, after which it was allowed to settle, for repeated water washings by decantation. This was followed by two washings with isopropyl alcohol, also by decantation. The green stones were then washed once with acetone to facilitate drying of the stones in a 40° C. oven.

Coloring of the Base Cream:

To a total of 80.00 g of base cream, in a PP beaker, was added 0.0179 g of Flavor Master Green Food Color (a blend of FD&C Yellow#5 and FD&C Blue#l). The mixture was stirred thoroughly with a PP spatula, until the color was completely homogeneous.

Addition of Exfoliants to Colored Cream:

To 80.00 g of green-colored cream, in a PP beaker, was added 1.60 g BaFAlSi (−100+200 mesh), 2.40 g BaFAlSi (−40+100 mesh), 0.40 g green-stained quince stone cells, and 0.40 g of ground green emerald stone (−40 mesh). The mixture was stirred vigorously with a PP spatula, until homogeneous.

Although the invention has been described and disclosed in various terms and certain embodiments, the scope of the invention is not intended to be, nor should it be deemed to be, limited thereby and such other modifications or embodiments as may be suggested by the teachings herein are particularly reserved, especially as they fall within the breadth and scope of the claims here appended. Those skilled in the art will recognize that these and other variations are possible within the scope of the invention as defined in the following claims and their equivalents.

What is claimed is:

1. An exfoliating cream comprising:
a base cream; and
particles of a stone,
wherein the virgin stone is of a characteristic color and wherein the exfoliating cream appears to be the color of the characteristic color.

2. The exfoliating cream of claim 1, wherein the stone is a precious or semi-precious stone.

3. The exfoliating cream of claim 2, wherein the stone is selected from the group consisting of topaz, emerald, amethyst, sapphire, agate, ruby, citrine, garnet, aquamarine, carnelian, fire opal, Peruvian opal, and turquoise.

4. The exfoliating cream of claim 1, further comprising particles of barium glass.

5. The exfoliating cream of claim 4, wherein the barium glass is a fluorine-containing barium glass

6. The exfoliating cream of claim 5, wherein the barium glass comprises barium, aluminum, fluorine, and silicon.

7. The exfoliating cream of claim 4, wherein the barium glass is the primary exfoliating agent.

8. The exfoliating cream of claim 1, further comprising whole stone cells.

9. The exfoliating cream of claim 1, further comprising at least one coloring agent to impart the characteristic color to the cream.

10. An exfoliating cream comprising:
a base cream; and
whole stone cells.

11. The exfoliating cream of claim 10, further comprising particles of ground barium glass.

12. The exfoliating cream of claim 11, wherein the barium glass is fluorine-containing.

13. The exfoliating cream of claim 10, wherein the stone cells are isolated from at least one of the fruits selected from the group consisting of pear, quince, and chico fruit.

14. The exfoliating cream of claim 13, further comprising at least one fragrance that imparts the smell of a fruit to the cream.

15. A method for forming an exfoliating cream, said method comprising the steps of:
providing a base cream;
providing particles of a stone to the base cream;
determining the color of the stone; and
providing at least one coloring agent to the base cream based on the color determination such that the exfoliating cream appears to be of the color of the stone.

16. The method of claim 15, wherein the stone is a precious or semi-precious stone.

17. The method of claim 16, wherein the stone is selected from the group consisting of topaz, emerald, amethyst, sapphire, agate, ruby, citrine, garnet, aquamarine, carnelian, fire opal, Peruvian opal, and turquoise.

18. The method of claim 17, further comprising providing particles of barium glass to the base cream.

19. The method of claim 18, wherein the barium glass is the primary exfoliating agent.

20. The method of claim 15, further comprising providing whole stone cells to the base cream.
